(19) United States
(12) Patent Application Publication
(10) Pub. No.: US 2022/0119751 A1
(43) Pub. Date: Apr. 21, 2022

(54) IMPELLER ASSEMBLY FOR A BIOPROCESSING SYSTEM

(71) Applicant: GLOBAL LIFE SCIENCES SOLUTIONS USA LLC, MARLBOROUGH, MA (US)

(72) Inventors: RYAN BARRETT, MARLBOROUGH, MA (US); JONATHAN KENNEY, MARLBOROUGH, MA (US)

(73) Assignee: GLOBAL LIFE SCIENCES SOLUTIONS USA LLC, MARLBOROUGH, MA (US)

(21) Appl. No.: 17/425,208

(22) PCT Filed: Apr. 29, 2020
(86) PCT No.: PCT/EP2020/061849

§ 371 Date: Jul. 22, 2021

Related U.S. Application Data
(60) Provisional application No. 62/841,855, filed on May 2, 2019.

(51) Int. Cl. C12M 1/06; C12M 1/00; B01F 27/054; B01F 27/808; B01F 33/453; B01F 35/21; B01F 35/513

(52) U.S. Cl. CPC C12M 27/02; C12M 23/14; C12M 23/26; B01F 35/513; B01F 27/808; B01F 33/453; B01F 35/2112; B01F 27/0543

(57) ABSTRACT

An impeller assembly for a bioprocessing system includes a hub and at least one blade pivotally to the hub, the at least one blade including a first leg portion and a second leg portion extending at an angle from the first leg portion. The at least one blade is rotatable between a first position where the first leg portion extends generally outwardly from the hub and a second position where the second leg portion extends generally outwardly from the hub.

### CROSS REFERENCE TO RELATED APPLICATIONS

This application is a national stage of International Application No. PCT/EP2020/061849 filed on Apr. 29, 2020, which claims priority to U.S. Provisional Patent Application No. 62/841,855 filed on May 2, 2019, all of which are hereby incorporated by reference in their entireties.

### BACKGROUND

### Technical Field

Embodiments of the invention relate generally to bioprocessing systems and methods and, more particularly, to an impeller assembly for a bioprocessing system.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

Increasingly, in the biopharmaceutical industry, single use or disposable containers are used. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. Depending on the fluid being processed, the system may include a number of fluid lines and different sensors, probes and ports coupled with the bag for monitoring, analytics, sampling, and fluid transfer. For example, a plurality of ports may typically be located at the front of the bag and accessible through an opening in the sidewall of the vessel, which provide connection points for sensors, probes and/or fluid sampling lines. In addition, a harvest port or drain line fitting is typically located at the bottom of the disposable bag and is configured for insertion through an opening in the bottom of the vessel, allowing for a harvest line to be connected to the bag for harvesting and draining of the bag after the bioprocess is complete.

Typically, an agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel. Magnetic coupling of the agitator inside the bag, to a drive system or motor external to the bag and/or bioreactor vessel, can eliminate contamination issues, allow for a completely enclosed system, and prevent leakage. Because there is no need to have a drive shaft penetrate the bioreactor vessel wall to mechanically spin the agitator, magnetically coupled systems can also eliminate the need for having seals between the drive shaft and the vessel.

Existing single-use, flexible bioprocessing bags and associated support vessels are available in a variety of sizes ranging from, for example, 50 liters to 2500 liters. These volumes indicate the approximate maximum operating volume of the bioprocessing system. Such systems are also operable at less than the maximum operating volumes, down to a minimum operating volume which is typically a function of the height of the impeller. For example, a 50 liter mixer system may be operable down to about 17 liters, and a 2500 liter mixer system may be operable down to about 520 liters. In certain situations, users may wish to operate a volumes less than the stated minimum operating volumes of the system. Existing bioprocessing system, however, are not capable of effective use at less than the stated minimum operating volumes.

In view of the above, there is a need for an impeller assembly for a bioprocessing system that facilitates operation of the system at volumes lower than current stated minimum operating volumes.

### BRIEF DESCRIPTION

In one aspect, an impeller assembly for a bioprocessing system includes a hub and at least one blade pivotally to the hub, the at least one blade including a first leg portion and a second leg portion extending at an angle from the first leg portion. The at least one blade is rotatable between a first position where the first leg portion extends generally outwardly from the hub and a second position where the second leg portion extends generally outwardly from the hub.

In one embodiment, an impeller assembly for a bioprocessing system includes a hub and at least one blade operatively connected to the hub and extending generally outwardly from the hub, wherein the impeller assembly has a height of about 39.9 millimeters to about 44.1 millimeters, and wherein the bioprocess system has a processing volume between about 50 liters and about 2500 liters.

In a second aspect, the invention discloses a flexible bioprocessing bag comprising the impeller assembly as discussed above. The bioprocessing bag can be used as a single-use bioreactor and has the advantage that it can be operated at both high and low operating volumes.

In a third aspect, the invention discloses a bioreactor comprising the above flexible bioprocessing bag mounted in and supported by a rigid support vessel.

In a fourth aspect, the invention discloses a method of operating the impeller assembly as discussed above, wherein a rotation direction of the impeller assembly is changed when an operational parameter has reached a predetermined value.

### DRAWINGS

The present invention will be better understood from reading the following description of non-limiting embodiments, with reference to the attached drawings, wherein below:

FIG. 1 is a front elevational view of a bioreactor system according to an embodiment of the invention.

FIG. 2 is a simplified side elevational, cross-sectional view of the bioreactor system of FIG. 1.

FIG. 3 is a perspective view of an impeller assembly according to another embodiment of the invention.

FIG. 4 is a schematic illustration of the impeller assembly of FIG. 5, showing a first mode of operation.

FIG. 5 is a schematic illustration of the impeller assembly of FIG. 5, showing a second mode of operation.

FIG. 6 is a schematic illustration of an impeller assembly with blades having a depending leg portion. a) side view of a blade, counterclockwise rotation, b) side view of a blade, clockwise rotation, c) front view of a blade, counterclockwise rotation, d) front view of a blade, clockwise rotation.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems, e.g., chromatography and tangential flow filter systems, and their associated flow paths. As used herein, the term “bag” means a flexible or semi-rigid container or vessel used, for example, as a bioreactor or mixer for the contents within.

Embodiments of the invention provide bioreactor or bioprocessing systems and impeller assemblies for a bioreactor or bioprocessing system. In an embodiment, an impeller assembly for a bioprocessing system includes a hub and at least one blade pivotally to the hub, the at least one blade including a first leg portion and a second leg portion extending at an angle from the first leg portion. The at least one blade is rotatable between a first position where the first leg portion extends generally outwardly from the hub and a second position where the second leg portion extends generally outwardly from the hub.

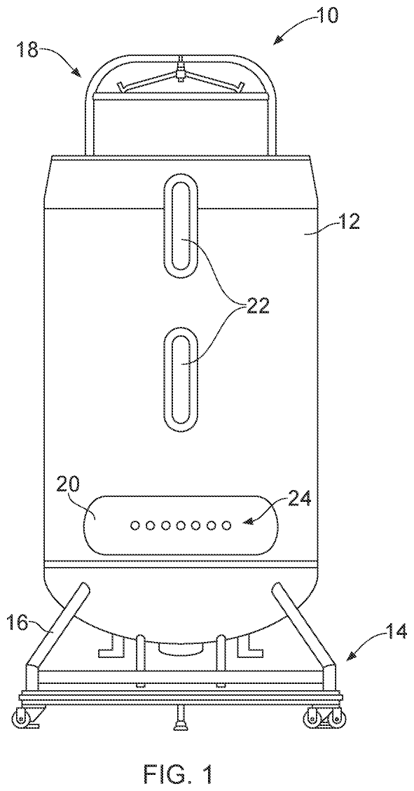

With reference to FIG. 1, a bioreactor system 10 according to an embodiment of the invention is illustrated. The bioreactor system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000 L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

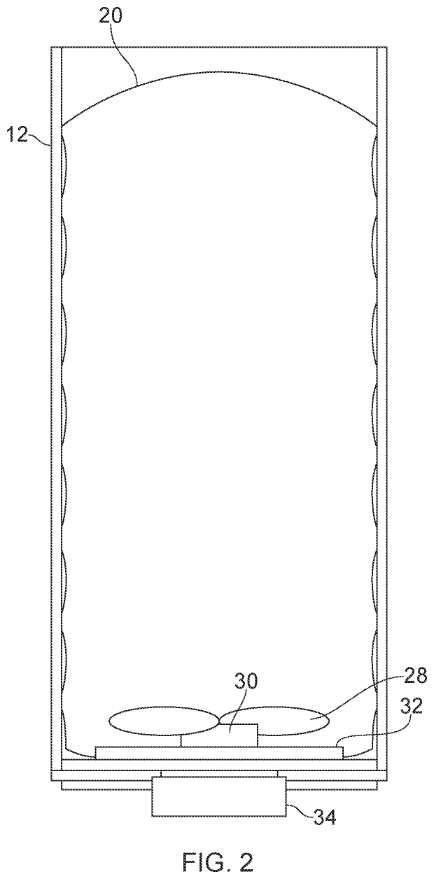

With specific reference to FIG. 2, a schematic side elevational, cutaway view of the bioreactor system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™, Bioclear™ 10 and Bioclear 11 laminates, available from GE Healthcare Life Sciences. Portions of the flexible container can comprise a substantially rigid material such as a rigid polymer, for example, high density polyethylene, metal, or glass. The flexible bag may be supplied pre-sterilized, such as using gamma irradiation. The bag can e.g. have a processing volume between about 10 liters and about 2500 liters, such as 50-2500 liters.

The flexible bag 20 contains an impeller 28 attached to a magnetic hub 30, suitably comprising one or more permanent magnets, at the bottom center of the inside of the bag, which rotates on an impeller plate 32 also positioned on the inside bottom of the bag 20. Together, the impeller 28 and hub 30 (and in some embodiments, the impeller plate 32) form an impeller assembly. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven impellers. A sparger (not shown) can suitably be located below the impeller, either integrated in the impeller plate or as a separate unit between the impeller plate (or a bottom wall of the bag) and the impeller. Bubbles from the sparger will then be dispersed by the impeller to achieve efficient aeration of a cell culture in the bioreactor.

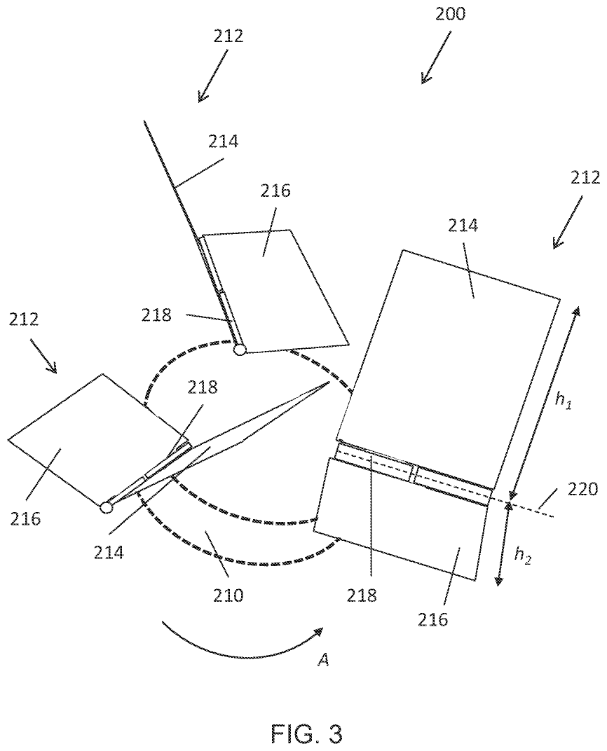

Referring now to FIG. 3, an impeller assembly 200 according to another embodiment of the invention is shown. The impeller assembly 200 includes a hub 210 and at least one blade 212 connected to the hub 210. The hub can be rotatably attached to a wall, such as a bottom wall, of the flexible bioprocessing bag 20, optionally via an impeller plate attached to the wall or bottom wall. The hub 210 is rotatable about a vertical axis that extends through the center of the hub 210. In an embodiment, the hub 210 may be a magnetic hub configured to be driven by the magnetic drive system or motor (e.g., motor 34 of FIG. 2) positioned exterior to the flexible bag 20 and vessel 12. While the impeller assembly 200 is shown in FIG. 3 as having three blades 212, the impeller assembly 200 may have fewer than three blades (e.g., one blade or two blades) or more than three blades (e.g. four, five or six blades), without departing from the broader aspects of the invention. The blades 212 may be equally spaced from one another about the hub 210. For example, where the impeller assembly 200 has three blades 212, the blades 112 may be spaced 120° apart.

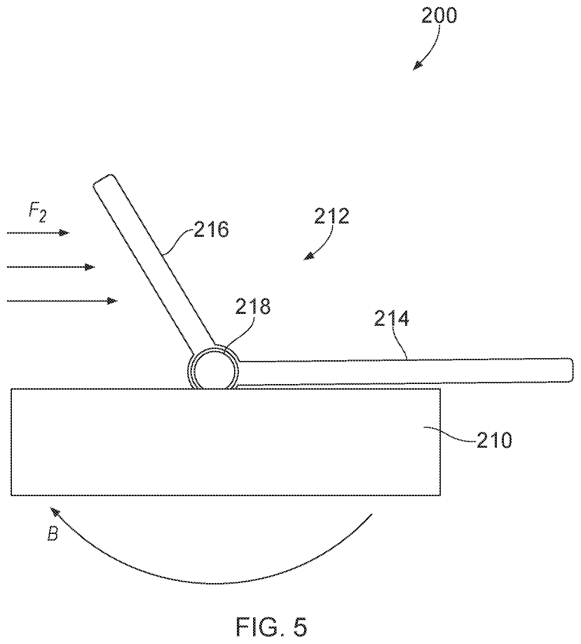

The blades 212 each include a first leg portion 214 and a second leg portion 216 positioned at an angle with respect to the first leg portion 214. The second leg portion may have a height, h2, that is less than the height, h1, of the first leg portion. The ratio h1:h2 may e.g. be 1.2-3, such as 1.5-2.5. As also shown in FIG. 5, each of the blades 212 is pivotally connected to the hub 210 via a shaft 218 that extends from the hub 210. Shaft 218 is shown as being generally horizontal but it can also be inclined or generally vertical. The shaft can e.g. be essentially parallel with a top, side or inclined surface of the hub. The blades 212 are connected to the hub 210 in such a manner that the blades 212 are permitted to rotated about an axis 220 of the horizontal shaft 218. While a shaft 218 may be one manner of pivotally connecting the blades 212 to the hub other means and mechanisms that provide for a pivoting action are also possible, such as a living hinge or flexible material.

While FIG. 3 shows that the first leg portion 214 and second leg portion 216 have different heights, in some embodiments the first leg portion 214 and the second leg portion 216 may have different configurations or geometries (with the same or different heights). More broadly, the first and second leg portions 214, 216 have different configurations from one another so as to provide different mixing characteristics, as discussed hereinafter.

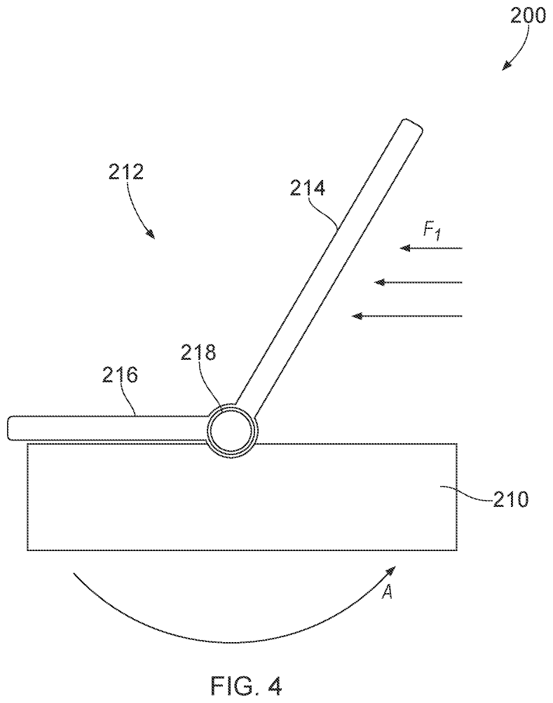

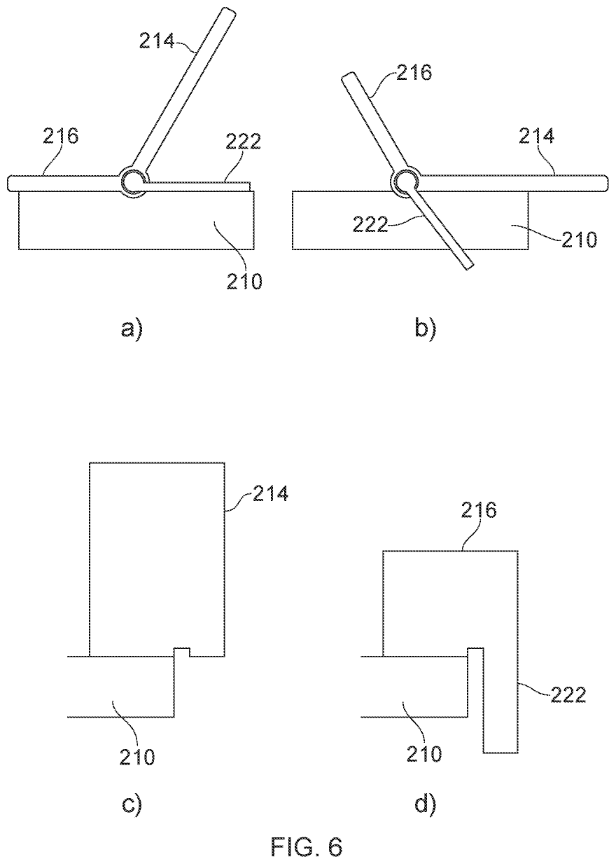

Turning now to FIGS. 4 and 5, operation of the impeller assembly 200 is shown. As illustrated in FIG. 4, when the impeller is rotated in a first direction, indicated by arrow A, the blades 212 move against the fluid within the flexible bag 20. The fluid, therefore, exerts a force, F₁, on the blades 212, which causes them to rotate about the shaft 218 to the position shown in FIG. 6. In this position, the taller leg portion 214 of each blade 212 extends generally outwardly (e.g., axially and/or radially) and is utilized for mixing the fluid within the bag 20.

As illustrated in FIG. 5, the impeller may also be rotated in a second, opposite direction, indicated by arrow B. When rotated in this direction, the blades 212 move against the fluid within the flexible bag 20, and the fluid exerts a force, F₂, on the blades 212, which causes them to rotate about the shaft 218 to the position shown in FIG. 5. In this position, the shorter leg portion 216 of each blade 212 extends generally outwardly (e.g., axially and radially) and is utilized for mixing the fluid within the bag 20.

In this respect, the direction of rotation of the impeller assembly 200 may be chosen to control which leg portion (i.e., the short leg portion 216 or the taller leg portion 214) is used for mixing. Accordingly, at when mixing or processing at a low volume is desired, the impeller may be rotated in a direction that causes the shorter leg portion 216 to extend upwardly for mixing the fluid. As the processing volume is increased, the direction of rotation of the impeller may be switched, causing the longer leg portion 214 to extend upwardly, for mixing the fluid. Essentially, therefore, the height of the impeller assembly 200 (i.e., the vertical height to the distal tip of the highest-extending blade portion) can be varied simply by rotating the impeller assembly 200 in different directions.

In an embodiment, illustrated by FIG. 6, each of the blades 212 (and one or both of the first leg portion 214 and second leg portion 216) may have a depending leg portion 222 that extends downwardly adjacent to the periphery of the hub 210. This depending leg portion may be utilized to mix the fluid below the upper surface of the hub 210, and may enable processing at even lower minimum operating volumes than have heretofore been possible.

The impeller assembly of the invention therefore allow for existing bioreactor systems to be operated at lower minimum operating volumes than has heretofore been possible. As indicated above, the minimum operating volume of a bioreactor system is dependent on the height of the impeller. Therefore, by utilizing a low-profile impeller, or by selectively controlling the height of the impeller blade utilized to mix the contents of the flexible bag, lower minimum operating volumes can be achieved in existing bioreactor vessels.

While the invention disclosed herein is described as a way to change the blade of the impeller based on the volume mixed, the blades can be changed (by altering the direction of rotation of the hub) in dependence upon any two desirable modes of mixing, e.g., fast/slow, thin/thick liquids, etc. That is, the position of the blades can be varied (by changing the direction of rotation of the impeller) to more broadly provide two different modes of mixing in a single impeller assembly. For example, the different modes may be high volume/low volume modes, or two different fluid viscosities/mediums (e.g. a two part mixture where part A is thicker and needs to be mixed before adding part B which is a thinner liquid or is a powder).

The rotation direction of the impeller assembly can advantageously be changed when an operational parameter has reached a predetermined value, e.g. when the volume of liquid in the vessel or flexible bioprocessing bag has reached a certain level. The liquid level can be measured e.g. if the bioreactor is mounted on load cells and the load cell signal can be sent to a control unit which controls the rotation speed and direction of the impeller. Alternatively, the operational parameter can be the viscosity of a liquid in the vessel/bag or a cell culture parameter for a cell culture in the vessel bag, such as a cell density or a viable cell density. This is advantageous for controlling agitation in a cell culture that starts at a low cell density and where the cell density increases with time, leading to a significant increase of the culture viscosity.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. As used herein to describe the present invention, directional terms such as “up”, down”, “upwards”, “downwards”, “upper”, “lower”, “top”, “bottom”, “vertical”, “horizontal”, “above”, “below” as well as any other directional terms, refer to those directions in the appended drawings.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. An impeller assembly for a bioprocessing system, comprising:
a hub; and
at least one blade pivotally connected to the hub, the at least one blade including a first leg portion and a second leg portion extending at an angle from the first leg portion;
wherein the at least one blade is rotatable between a first position where the first leg portion extends generally outwardly from the hub and a second position where the second leg portion extends generally outwardly from the hub.

2. The impeller assembly of claim 1, wherein:
the first leg portion has a height that is greater than a height of the second leg portion.

3. The impeller assembly of claim 1, wherein:
the first leg portion has a height that is 1.2-3 times the height of the second leg portion, such as 1.5-2.5 times the height of the second leg portion.

4. The impeller assembly of claim 1, wherein:
the at least one blade is pivotally connected to the hub via a shaft that extends from the hub.

5. The impeller assembly of claim 4, wherein:
said shaft is essentially parallel with a top, side or inclined surface of the hub.

6. The impeller assembly of claim 4, wherein:
said shaft is a horizontal shaft.

7. The impeller assembly of claim 1, wherein:
the at least one blade is pivotally connected to the hub via a living hinge.

8. The impeller assembly of claim 1, wherein:
the at least one blade is three blades, four blades, five blades or six blades.

9. The impeller assembly of claim 1, wherein said at least one blade is configured to pivot between the first and second position upon a change in a rotation direction of the hub.

10. A flexible bioprocessing bag comprising the impeller assembly of claim 1.

11. The flexible bioprocessing bag of claim 10, wherein said hub is rotatably attached to a wall, such as a bottom wall, of said flexible bioprocessing bag, optionally via an impeller plate attached to said wall or bottom wall.

12. The flexible bioprocessing bag of claim 11, further comprising a sparger mounted between said hub and said wall, bottom wall or impeller plate.

13. The flexible bioprocessing bag of claim 11, further comprising a sparger mounted in said impeller plate.

14. The flexible bioprocessing bag of claim 10, wherein said hub comprises a plurality of magnets and wherein said impeller assembly is configured to be magnetically driven, such as by an external magnetic drive.

15. The flexible bioprocessing bag of claim 10, wherein said bag is presterilized, such as by gamma irradiation.

16. The flexible bioprocessing bag of claim 10, wherein said bag has a processing volume between about 10 liters and about 2500 liters, such as 50-2500 liters.

17. A bioreactor, comprising the flexible bioprocessing bag of claim 10, mounted in and supported by a rigid support vessel.

18. The bioreactor of claim 17, wherein said rigid support vessel comprises a magnetic drive configured to drive said impeller assembly.

19. A method of operating the impeller assembly of claim 1, wherein a rotation direction of said impeller assembly is changed when an operational parameter has reached a predetermined value.

20. The method of claim 19, wherein said operational parameter is a volume of liquid in a vessel or flexible bioprocessing bag wherein said impeller assembly is mounted.

21. The method of claim 19, wherein said operational parameter is a viscosity of a liquid in a vessel or flexible bioprocessing bag wherein said impeller assembly is mounted.

22. The method of claim 19, wherein said operational parameter is a cell culture parameter, such as a cell density or a viable cell density of a cell culture in a vessel, flexible bioprocessing bag or bioreactor wherein said impeller assembly is mounted.

23. An impeller assembly for a bioprocessing system, comprising:
a hub; and
at least one blade operatively connected to the hub and extending generally outwardly from the hub;
wherein the impeller assembly has a height of about 39.9 millimeters to about 44.1 millimeters; and
wherein the bioprocess system has a processing volume between about 50 liters and about 2500 liters.
